(19) United States
(12) Patent Application Publication
(10) Pub. No.: US 2016/0081583 A1
(43) Pub. Date: Mar. 24, 2016

(54) POSITION DETECTING APPARATUS AND POSITION DETECTING METHOD FOR MEDICAL DEVICE AND ENDOSCOPE APPARATUS

(71) Applicant: OLYMPUS CORPORATION, Tokyo (JP)

(72) Inventors: Soichi IKUMA, Akishima-shi (JP); Jun HASEGAWA, Hino-shi (JP)

(73) Assignee: OLYMPUS CORPORATION, Tokyo (JP)

(21) Appl. No.: 14/960,851

(22) Filed: Dec. 7, 2015

Related U.S. Application Data
(63) Continuation of application No. PCT/JP2014/062857, filed on May 14, 2014.

(30) Foreign Application Priority Data
Jun. 7, 2013 (JP) 2013-121017

(51) Int. Cl. A61B 5/06; A61B 1/005; A61B 1/00

(52) U.S. Cl. CPC A61B 5/062; A61B 1/00006; A61B 1/005

(57) ABSTRACT

A position detecting apparatus for a medical device includes a transmission unit configured to transmit a magnetic field in each of a plurality of frame periods. A reception unit detects the magnetic field transmitted from the transmission unit and output a detection signal corresponding to a position of the transmission unit. A signal control unit causes the transmission unit to transmit the magnetic field by a multiplexed signal including at least two or more different frequencies in a certain frame period among the frame periods, and causes the transmission unit to transmit the magnetic field by the multiplexed signal in the frame periods other than the certain frame period when the detection signal output from the reception unit has changed to a certain threshold or higher.

### CROSS-REFERENCE TO RELATED APPLICATIONS

This application is a Continuation Application of PCT Application No. PCT/JP2014/062857, filed May 14, 2014 and based upon and claiming the benefit of priority from the prior Japanese Patent Application No. 2013-121017, filed Jun. 7, 2013, the entire contents of both of which are incorporated herein by reference.

### BACKGROUND OF THE INVENTION

1. Field of the Invention

The present invention relates to a position detecting apparatus and a position detecting method for a medical device configured to detect the shape of the medical device, such as an endoscope, inserted in a subject, and to an endoscope apparatus using the position detecting apparatus.

2. Description of the Related Art

There has been known an endoscope position detecting apparatus, which detects the shape and other properties of an endoscope inserted in a subject such as a patient. Such an apparatus includes a plurality of transmitting coils disposed on an insertion member of the endoscope and a plurality of receiving coils as an antenna AT. The apparatus transmits magnetic fields from the transmitting coils in a time divisional manner and receives the transmitted magnetic fields with the receiving coils as the antenna AT to detect the positions of the transmitting coils, thereby detecting the shape of the insertion member of the endoscope on the basis of the positions.

In an environment, such as an operating room, where an endoscope is used, various medical devices, for example, a fluoroscope, an operating table, and a bed for a patient are installed. Such medical devices include metallic members. In the above-described environment, the insertion member of the endoscope is inserted in a subject such as a patient, and the shape of the insertion member inserted in the subject, is detected.

When the transmitting coils transmit magnetic fields for detecting the shape and other properties of the insertion member inserted in the subject such as a patient, the magnetic fields generate eddy currents in the metallic members in the various medical devices, and the eddy currents generate secondary magnetic fields. The eddy currents become larger as the frequencies of the magnetic fields transmitted from the transmitting coils become higher. The magnetic fields generated by the eddy currents tend to cause disturbance in the transmission and the reception of magnetic fields between the transmitting coils and the receiving coils of the apparatus.

For example, Jpn. PCT National Publication No. 2006-523473 (WO2004/091391A1) has been known as a technique to correct such disturbance due to magnetic fields. Jpn. PCT National Publication No. 2006-523473 (WO2004/091391A1) discloses that a computer system analyzes position indication signals generated at multiple frequencies, calculates an eddy current phase and eddy current amplitude on the basis of the position indication signals, and removes measurement errors induced by eddy currents by correcting position indication.

### BRIEF SUMMARY OF THE INVENTION

According to a first aspect of the invention, there is provided a position detecting apparatus for a medical device, comprising: a transmission unit configured to transmit a magnetic field in each of a plurality of frame periods; a reception unit configured to detect the magnetic field transmitted from the transmission unit and output a detection signal corresponding to a position of the transmission unit; and a signal control unit configured to cause the transmission unit to transmit the magnetic field by a multiplexed signal including at least two or more different frequencies in a certain frame period among the frame periods, and cause the transmission unit to transmit the magnetic field by the multiplexed signal in the frame periods other than the certain frame period when the detection signal output from the reception unit has changed to a certain threshold or higher.

According to a second aspect of the invention, there is provided a position detecting apparatus for a medical device, comprising: a transmission unit including a plurality of coils and configured to transmit a magnetic field in each of a plurality of frame periods; a reception unit configured to detect the magnetic field transmitted from the transmission unit and output a detection signal corresponding to each of positions of the coils; and a signal control unit configured to cause the transmission unit to transmit the magnetic field by a multiplexed signal including at least two or more different frequencies from each of the coils on the basis of a positional relation of the coils.

According to a third aspect of the invention, there is provided a position detecting method for a medical device, comprising: transmitting a magnetic field from a plurality of coils in each of a plurality of consecutive frame periods; receiving the transmitted magnetic field by a reception unit and outputting a detection signal corresponding to each of positions of the coils; and causing, by a signal control unit, the transmission unit to transmit the magnetic field by a multiplexed signal including at least two or more different frequencies from each of the coils on the basis of a positional relation of the coils.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute apart of the specification, illustrate embodiments of the invention, and together with the general description given above and the detailed description of the embodiments given below, serve to explain the principles of the invention.

FIG. 1 is a configuration diagram illustrating an embodiment of a position detecting apparatus for a medical device according to the present invention.

FIG. 2 is a configuration diagram illustrating an endoscope apparatus to which the apparatus in FIG. 1 is applied.

FIG. 3 is a diagram illustrating an arrangement of a plurality of transmitting coils in an insertion member 20 in the apparatus.

FIG. 4 is a diagram illustrating transmission timing in a first frame period of the apparatus in a first embodiment.

FIG. 5 is a diagram illustrating transmission timing in a second frame period of the apparatus.

FIG. 6 is a diagram illustrating transmission timing in a third frame period or later of the apparatus.

FIG. 7 is a diagram illustrating transmission timing in an odd-numbered frame period of the apparatus in a second embodiment.

FIG. 8 is a diagram illustrating transmission timing in an even-numbered frame period of the apparatus.

### DETAILED DESCRIPTION OF THE INVENTION

### First Embodiment

The following describes the first embodiment of the present invention with reference to the accompanying drawings.

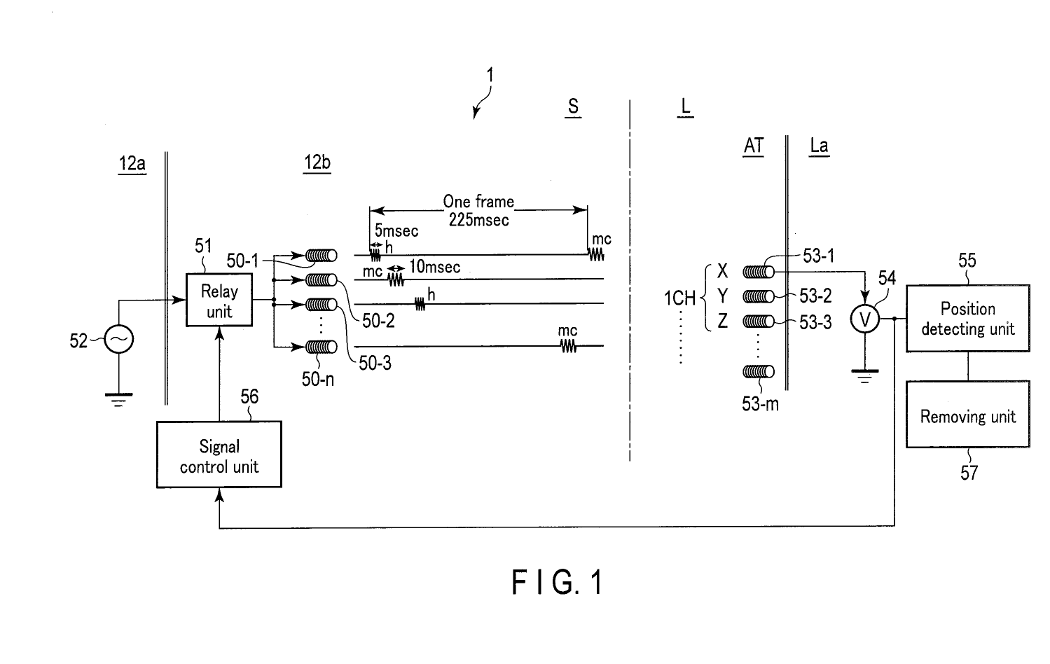

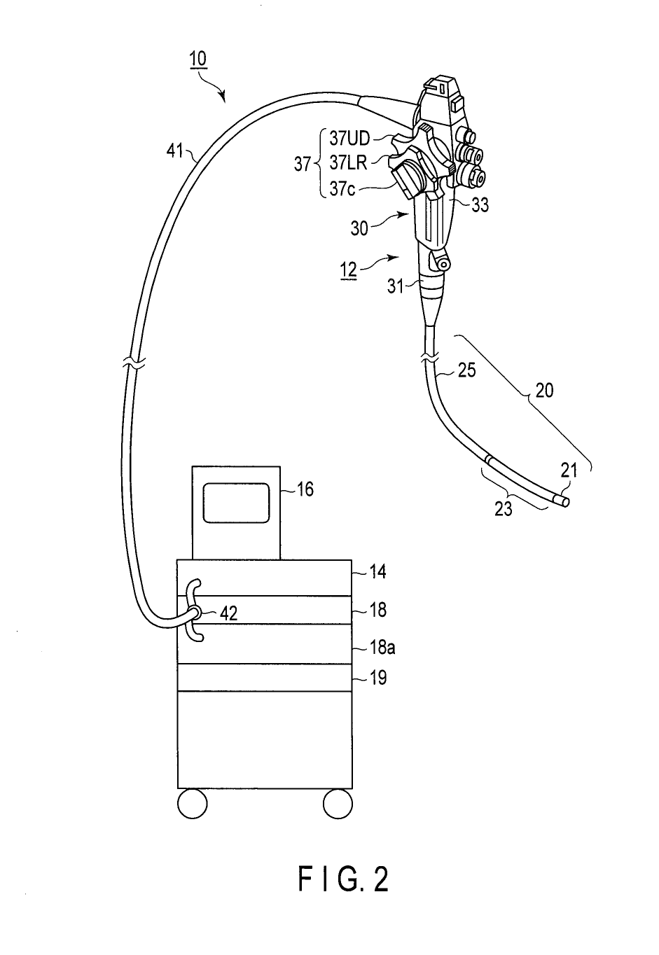

FIG. 1 is a configuration diagram of a position detecting apparatus 1 (hereinafter referred to as the apparatus) for a medical device. The apparatus 1 is provided to a medical device, for example, an endoscope apparatus (tubular insertion system) 10 illustrated in FIG. 2. The endoscope apparatus 10 includes an insertion member 20 configured to be inserted in a body cavity (lumen) of, for example, a patient as a subject to observe or treat an affected part or lesioned part in the body cavity. The apparatus 1 detects the position, the shape, and other attributes of the insertion member 20 of the endoscope apparatus 10 inserted in the body cavity. The apparatus 1 is applicable for detecting the position, the shape, and other attributes of forceps and a catheter used as medical devices in the endoscope apparatus 10, not limited to the endoscope apparatus 10.

FIG. 1 illustrates the apparatus 1 that includes a transmission unit S and a reception unit L. The transmission unit S includes a body-side part 12a and a scope-side part 12b in the endoscope 12 of the endoscope apparatus 10. The reception unit L includes an antenna AT and a receiver-side part La.

As illustrated in FIG. 2, the endoscope apparatus 10 includes an endoscope 12, an image processor 14 (for example, a video processor), a monitor 16, a light source 18, an inserted shape estimating unit 18a, and a controller 19.

The endoscope 12 images the inside of the body cavity (lumen) of a patient.

Image processor 14 subjects an image the inside of the body cavity of the patient imaged by the endoscope 12 to image processing.

The monitor 16 is connected to the image processor 14. The monitor 16 displays the image in the body cavity of the patient that has been imaged by the endoscope 12 and subjected to image processing by the image processor 14.

The light source 18 emits light as illumination light to be projected from the endoscope 12.

The inserted shape estimating unit 18a supplies transmitting coils 50-1 to 50-n (n: natural number) with electric power, detects voltages on a plurality of receiving coils 53-1 to 53-m (m: natural number) that are components of the antenna AT, and estimates the shape of the insertion member 20 inserted in the body cavity of the patient on the basis of the detected voltages.

The controller 19 controls the entire endoscope apparatus 10 including the endoscope 12, the image processor 14, the monitor 16, the light source 18, and the inserted shape estimating unit 18a.

The endoscope 12 includes the insertion member 20 and an operation unit 30. The insertion member 20 is configured to be inserted in a body cavity of a patient. The operation unit 30 is configured to be operated to control the endoscope 12 and is connected to a proximal end member of the insertion member 20.

The insertion member 20 is formed in an elongated hollow tube. In the insertion member 20, a distal rigid portion 21, a curving portion 23, and a flexible tubular portion 25 are continuously formed from a distal end toward a proximal end. The distal rigid portion 21 is formed of a rigid material. A proximal end of the distal rigid portion 21 connects to a distal end of the curving portion 23. The distal rigid portion 21 is a distal end of the insertion member 20 and the endoscope 12.

The curving portion 23 is formed to be flexible. A proximal end of the curving portion 23 connects to a proximal end of the flexible tubular portion 25. The curving portion 23 curves to a desired direction, for example, upward, downward, rightward, or leftward, in response to an operation on a curve operation member 37 by an operator. The position and the direction of the distal rigid portion 21 are changed by the curving of the curving portion 23. With this configuration, an image in the body cavity of the patient is captured in an observation visual field of the endoscope 12. In addition, the inside of the body cavity of the patient is irradiated with the illumination light projected from the endoscope 12. The curving portion 23 includes joint rings arranged along the longitudinal direction of the insertion member 20 and connected to each other in a rotatable manner.

The flexible tubular portion 25 is formed of a flexible material. The flexible tubular portion 25 is curved by external force applied thereto. The flexible tubular portion 25 is a tubular member extending from a body 31 in the operation unit 30.

The operation unit 30 includes the body 31, a handle 33, and a universal cord 41. The body 31 is a member from which the flexible tubular portion 25 extends. The handle 33 is connected to a proximal end of the body 31 and configured to be held by an operator of the endoscope 12. The universal cord 41 is connected to the handle 33.

On the handle 33, a curve operation member 37 for operating an operation wire to curve the curving member 23 is disposed. The curve operation member 37 includes an upward/downward curve operation knob 37UD, a rightward/leftward curve operation knob 37LR, and a fixing knob 37c. The upward/downward curve operation knob 37UD curves the curving portion 23 in the upward or the downward direction. The rightward/leftward curve operation knob 37LR curves the curving portion 23 in the rightward or the leftward direction. The fixing knob 37c fixes the position of the curving portion 23 having curve shape.

The universal cord 41 extends from a side surface of the handle 33 of the endoscope 12 and connects the handle 33, the image processor 14, the light source 18, and the inserted shape estimating unit 18a to each other. The universal cord 41 mediates data exchange between the handle 33 and the image processor 14, the light source 18, and the inserted shape estimating unit 18a. The universal cord 41 includes a connector 42 disposed on a proximal end. The connector 42 is removable from the image processor 14, the light source 18, and the inserted shape estimating unit 18a.

The image processor 14, the light source 18, the inserted shape estimating unit 18a, and the controller 19 are electrically connected to each other. The image processor 14, the light source 18, and the inserted shape estimating unit 18a are removably connected to the endoscope 12 through the connector 42.

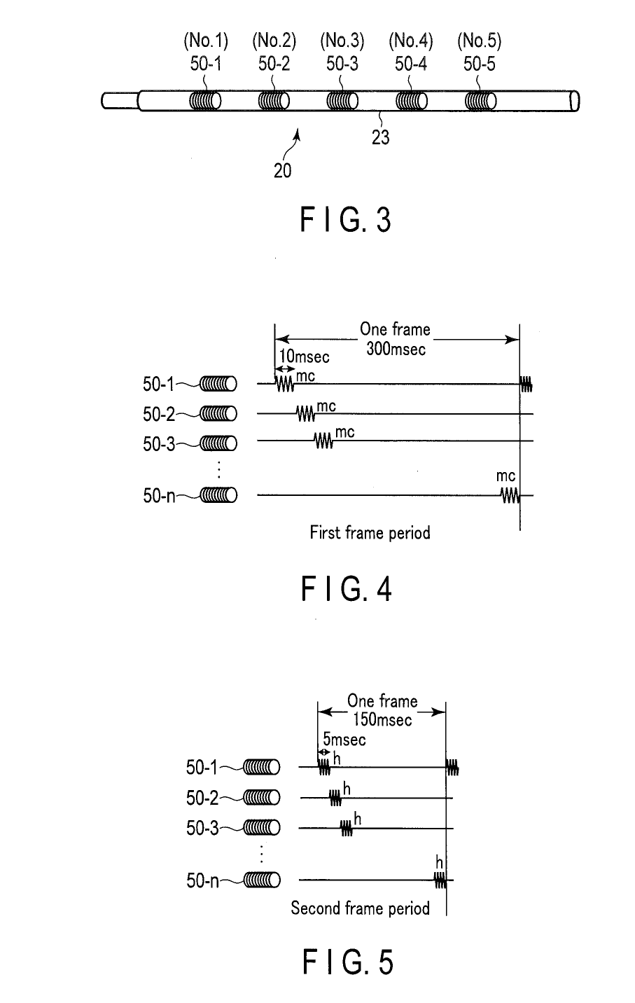

In the insertion member 20 of the above-described endoscope apparatus 10, a plurality of transmitting coils 50-1 to 50-n illustrated in FIG. 3, for example, thirty transmitting coils (n=30) are disposed at certain intervals. The transmitting coils 50-1 to 50-n each are supplied with electric power to generate magnetic fields (electromagnetic fields), and transmit the generated magnetic fields. For example, the transmitting coil 50-1 is defined as No. 1; the transmitting coil 50-2 as No. 2; . . . and the transmitting coil 50-n as No. n, in the order of the transmitting coils 50-1 to 50-n.

The transmitting coils 50-1 to 50-n are connected to a transmitter power supply 52 through a relay unit 51 to selectively supply electric power. For example, the relay unit 51 is disposed on the connector 42 or the operation unit 30. For example, the relay unit 51 includes a plurality of relays connected to the respective transmitting coils 50-1 to 50-n. The relay unit 51 turns on the relays corresponding to the transmitting coils 50-1 to 50-n to transmit magnetic fields, to supply the respective transmitting coils 50-1 to 50-n with alternating current power from the transmitter power supply 52 through the relays.

For example, the transmitter power supply 52 is included in the inserted shape estimating unit 18a or other parts. The transmitter power supply 52 outputs alternating current powers of at least two or more different frequencies, for example, a first frequency f1 (=400 Hz) and a second frequency f2 (=800 Hz). For example, the electric power from the transmitter power supply 52 is supplied to the transmitting coils 50-1 to 50-n through the relay unit 51 with a plurality of power lines disposed in the universal cord 41. The transmission unit S includes the transmitting coils 50-1 to 50-n, the relay unit 51, the transmitter power supply 52, and other components.

The receiving coils 53-1 to 53-m are components of the antenna AT. The receiving coils 53-1 to 53-m, for example, twelve receiving coils (m=12) are disposed outside a subject such as a patient. For example, the receiving coils 53-1 to 53-m are disposed in an examination room or an operation room. The inside of the body cavity of the subject such as a patient lying on a bed may be observed or treated with the insertion member 20 inserted in a body cavity of the patient. In this case, the receiving coils 53-1 to 53-m are disposed near the bed and in a range in which the magnetic fields transmitted from the transmitting coils 50-1 to 50-n are detectable.

For example, the total number of the receiving coils 53-1 to 53-m is 12, specifically, four coils the axes of which are aligned in the x direction, four coils the axes of which are aligned in the y direction, and four coils the axes of which are aligned in the z direction. The receiving coils 53-1 to 53-m each are disposed at a different position in the antenna AT. The receiving coils 53-1 to 53-m each detect, in the x, y, and z directions, the alternating magnetic fields transmitted from the transmitting coils 50-1 to 50-n, and generate the voltages corresponding to the magnitude of the magnetic fields in the x, y, and z directions, on both ends of each of the receiving coils 53-1 to 53-m.

The receiving coils 53-1 to 53-m each have an output terminal connected to a corresponding voltage detector 54. The voltage detectors 54 are included in the inserted shape estimating unit 18a. The voltage detectors 54 each detect the voltage generated between both ends of each of the receiving coils 53-1 to 53-m, perform frequency analysis, and determine amplitude and a phase for each frequency. The voltage detectors 54 output voltage detection signals that are sent to a removing unit 57. The removing unit 57 is included in the inserted shape estimating unit 18a.

The removing unit 57 calculates the amplitude of the transmitted wave from which the influence of eddy currents in external magnetic members (metals) has been removed. There are two kinds of amplitude calculation methods that depend on the type (mc or h) of the transmission wave of each of the transmitting coils 50-1 to 50-n.

First, when the type of the transmission wave from the transmitting coils 50-1 to 50-n is mc, the removing unit 57 calculates the amplitude from which the influence of eddy currents in external magnetic members (metals) has been removed on the basis of the amplitude and the phase of, for example, the first frequency f1 (=400 Hz) and the second frequency f2 (=800 Hz) with different magnitude of the magnetic fields due to eddy currents. The amplitude calculation methods are publicly known. For example, refer to WO01/33162 (Jpn. PCT National Publication No. 2003-513260) and WO2004/091391 (Jpn. PCT National Publication No. 2006-523473). The amplitude calculated for each frequency, along with a phase, is sent to the position detecting unit 55 included in the inserted shape estimating unit 18a.

Second, when the type of the transmission wave of the transmitting coils 50-1 to 50-n is h, the removing unit 57 calculates the amplitude from which the influence of eddy currents in external magnetic members (metals) has been removed on the basis of the amplitude and the phase of the second frequency f2 (=800 Hz) and the influence ratio of the last eddy currents. The influence ratio of the last eddy currents is a ratio of the amplitude before and after removing the influence of magnetic members (metals) external to the receiving coils 53-1 to 53-m in the last frame period among the past frame periods in which transmission waveforms of an mc type have been transmitted from the transmitting coils 50-1 to 50-n. The removing unit 57 transmits the calculated amplitudes with phases to the position detecting unit 55 included in the inserted shape estimating unit 18a.

The position detecting unit 55 detects the positions of the transmitting coils 50-1 to 50-n on the basis of the phase, and the amplitude from which the influence of eddy currents has been removed, of each frequency on each of the receiving coils 53-1 to 53-m, thereby detecting the shape of the insertion member 20 of the endoscope apparatus 10 on the basis of the position of each of the transmitting coils 50-1 to 50-n, for example, detecting the shape of the insertion member 20 inserted in a curve form or the like in a body cavity of a patient. The reception unit L includes the receiving coils 53-1 to 53-m, the voltage detectors 54, the removing unit 57, the position detecting unit 55, and other components.

When a piece of metal, which is an external magnetic member, exists in an environment in which magnetic fields are transmitted and received, disturbance due to magnetic components generated in the metal may occur in the transmission and reception of the magnetic fields. For example, in a metal existing in varying magnetic fields, eddy currents are generated by the varying magnetic fields, and secondary magnetic fields occur due to the eddy currents. When the receiving coils 53-1 to 53-m receive the secondary magnetic fields, voltage components by the magnetic fields transmitted from the transmitting coils 50-1 to 50-n and voltage components by the secondary magnetic fields are added to the voltage generated between both ends of each of the receiving coils 53-1 to 53-m. As a result, influence of the magnetic field components generated in external metals is applied to the magnitude of the magnetic fields received by the receiving coils 53-1 to 53-m.

Under the influence of the of the magnetic fields, the detection of the position of each of the transmitting coils 50-1 to 50-n by detecting the magnetic fields with the receiving coils 53-1 to 53-m may be inaccurate. In order to remove the influence of the magnetic fields due to eddy currents, the transmitting coils 50-1 to 50-n each need to transmit magnetic fields at multiple frequencies.

The multiple frequencies need to be relatively low frequencies, for example, several tens of hertz to several hundreds of hertz depending on the kinds of the external magnetic members (metals) existing in an environment in which magnetic fields are transmitted and received. The reason is that the phases of the received signals at low frequencies are largely influenced by eddy currents and the influence is easily detected. Because the phases are used in calculation for removing the influence by eddy currents, the influence by eddy currents can be removed accurately at the low frequencies. Such frequencies may have at least two or more different frequency values, for example, the first frequency f1 (=400 Hz) and the second frequency f2 (=800 Hz) higher than the first frequency f1.

The inserted shape estimating unit 18a includes a signal control unit 56 that is configured to on/off drive the relay unit 51, supply the transmitting coils 50-1 to 50-n with electric power from the transmitter power supply 52 through the relay unit 51, and transmit magnetic fields sequentially from the transmitting coils 50-1 to 50-n in a time divisional manner. Now, a period of time for completing the magnetic field transmission from all the transmitting coils 50-1 to 50-n is defined as one frame period.

The signal control unit 56 causes the transmitting coils 50-1 to 50-n to sequentially transmit a magnetic field mc by a multiplexed signal formed of a combined wave obtained by combining at least two or more frequencies, for example, the first frequency f1 (=400 Hz) and the second frequency f2 (=800 Hz), at certain regular intervals in a certain frame period, for example, in the first frame period.

For example, the magnetic field mc by the multiplexed signal is transmitted in a transmission period of 10 ms with a varied amplitude in four wavelengths. In order to estimate the influence of the magnitude of the magnetic fields due to eddy currents generated in external magnetic members (metals) existing in an environment in which magnetic fields are transmitted and received, the signal control unit 56 causes each of the transmitting coils 50-1 to 50-n to transmit the magnetic field mc by the multiplexed signal in the first frame period. For example, in the case of n=30, the length of a frame period is 300 ms.

The signal control unit 56 causes the transmitting coils 50-1 to 50-n to sequentially transmit only a magnetic field h by a single signal having the second frequency f2 (=800 Hz) at certain regular intervals in a frame period other than the certain frame period, for example, each frame period after the first frame period, by driving the relay unit 51.

The signal control unit 56 causes each of the transmitting coils 50-1 to 50-n to transmit the magnetic field mc by the multiplexed signal in a frame period other than the certain frame period, for example, each frame period after the first frame period, when the magnitude of the magnetic field detected by each of the receiving coils 53-1 to 53-m has changed, that is, the voltage of the detection signal output from each voltage detector 54 has changed to a certain threshold or higher. For example, also each magnetic field mc by the multiplexed signal is transmitted in a transmission period of 10 ms with a varied amplitude in four wavelengths.

The signal control unit 56 causes each of the transmitting coils 50-1 to 50-n to transmit only the magnetic field h by the single signal having the second frequency f2 (=800 Hz) when the magnitude of the magnetic field transmitted from each of the transmitting coils 50-1 to 50-n, that is, the voltage of a detection signal output from the voltage detectors 54 is not the certain threshold or higher. For example, each single signal is transmitted from the transmitting coils 50-1 to 50-n in a transmission period of 5 ms with a varied amplitude in four wavelengths.

The following describes a first determination method for determining whether the voltage of the detection signal corresponding to the magnitude of the magnetic fields is the certain threshold or higher. The first determination method is a determination method using a threshold of a change rate in the amplitude of a detection signal corresponding to the magnitude of the magnetic fields.

First, the transmitting coils 50-1 to 50-n sequentially transmit the magnetic field in a time divisional manner in the order of No. n (n=1, 2, . . . , 30).

Next, every time the respective transmitting coils 50-1 to 50-n sequentially transmit the magnetic field in a time divisional manner from No. n (n=1, 2, . . . , 30), the voltage detectors 54 analog/digital (A/D)-convert each voltage detection signal in a chronological order, perform the fast Fourier transform (FFT) analysis on a sequence of the A/D-converted digital detection signals, and calculate an amplitude and a phase for each of the frequencies including the first frequency f1 (=400 Hz) and the second frequency f2 (=800 Hz) both obtained by the FFT analysis.

Next, the signal control unit 56 sets the certain threshold to a voltage, for example, corresponding to 2% of the amplitude of the second frequency f2 (=800 Hz) output from the voltage detectors 54 when the receiving coils 53-1 to 53-m have received a magnetic field, for example, transmitted from the transmitting coil 50-1 as No. 1 in one frame period before the current frame period. The first frame period has no previous frame period, and therefore the signal control unit 56 does not set the threshold.

As described above, the signal control unit 56 causes each of the transmitting coils 50-1 to 50-n to transmit the magnetic field mc by the multiplexed signal in each frame period after the first frame period, when the magnitude of the magnetic field detected by each of the receiving coils 53-1 to 53-m, that is, the amplitude of the second frequency f2 (=800 Hz) output from each voltage detector 54 has changed to the certain threshold or higher.

Specifically, when the difference between a first amplitude of the second frequency f2 (=800 Hz) calculated by the FFT analysis and, for example, a second amplitude of the second frequency f2 (=800 Hz) calculated by the FFT analysis in one frame period before the current frame period (the first amplitude−the second amplitude) is the certain threshold or higher, the signal control unit 56 causes the magnetic field mc by the multiplexed signal formed of the combined wave obtained by combining the first frequency f1 (=400 Hz) and the second frequency f2 (=800 Hz) to be transmitted at the next timing of transmitting the magnetic field from the transmitting coil 50-1 as No. 1.

The second amplitude of the second frequency f2 (=800 Hz) is calculated by performing the FFT analysis on the signals corresponding to the magnitude of the magnetic fields detected by the receiving coils 53-1 to 53-m, that is, the detection signals output from the voltage detectors 54, when the magnetic field mc by the multiplexed signal is transmitted from the transmitting coil 50-1 as No. 1 and received by the receiving coils 53-1 to 53-m in one frame period before the current frame period.

The certain threshold is a voltage, for example, corresponding to 2% of the amplitude of the second frequency f2 (=800 Hz) calculated by the FFT analysis.

Next, the following describes a second determination method for determining whether the voltage of the detection signal corresponding to the magnitude of magnetic fields is the certain threshold or higher. The second determination method is a determination method using a threshold of a change amount in calculated positional information.

First, the transmitting coil 50-1 as No. 1 transmits the magnetic field, and substantially at the same time, the receiving coils 53-1 to 53-m receive the transmitted magnetic field. Thereafter, signal processing is performed as follows.

For example, the number of the disposed receiving coils 53-1 to 53-m is 12. The voltages corresponding to the magnitude of the magnetic fields generated on the receiving coils 53-1 to 53-m are parallelly processed.

Upon receiving the magnetic fields, the receiving coils 53-1 to 53-m each generate the voltages corresponding to the magnitude of the magnetic fields on both ends of each of the receiving coils 53-1 to 53-m.

The receiving coils 53-1 to 53-m each have an output terminal connected to corresponding one of the voltage detectors 54.

Each of the voltage detectors 54 produces a sequence of digital detection signals by A/D-converting a detection signal of a magnetic field in a chronological order every time the transmitting coils 50-1 to 50-n as No. n (n=1, 2, . . . , 30) sequentially transmit the magnetic field.

The voltage detector 54 calculates the amplitude and the phase for each of the first frequency f1 (=400 Hz) and the second frequency f2 (=800 Hz) by performing the FFT analysis on the series of the digital data of the voltages.

The removing unit 57 calculates an amplitude from which influence due to external magnetic members (metals) has been removed.

Next, the position detecting unit 55 calculates the positions of the transmitting coils 50-1 to 50-n and the directions of the axes of the transmitting coils 50-1 to 50-n (the directions of the coils) on the basis of the amplitudes at the twelve receiving coils 53-1 to 53-m from which the influence due to external magnetic members (metals) has been removed.

Then, for example, the signal control unit 56 sets the certain threshold to 5 mm.

Thereafter, the signal control unit 56 determines whether the difference between the position of the transmitting coil 50-1 and, for example, the position of the transmitting coil 50-1 that has transmitted the magnetic field mc by the multiplexed signal in one frame period before the current frame period is the certain threshold (5 mm) or larger. When the determination result shows that the difference between the positions of the coils is the certain threshold (5 mm) or larger, the signal control unit 56 causes the magnetic field mc by the multiplexed signal formed of the combined wave obtained by combining the first frequency f1 (=400 Hz) and the second frequency f2 (=800 Hz) to be transmitted at the next timing of transmitting the magnetic field from the transmitting coil 50-1 as No. 1.

Thereafter, the transmitting coils 50-1 as No. 2, No. 3, . . . , and No. m sequentially transmit the magnetic field in a time divisional manner, and at each time of the magnetic field transmission, the similar processing described above is performed. The signal control unit 56 determines whether the differences between the positions of the transmitting coils 50-2 to 50-n and, for example, the positions of the transmitting coils 50-2 to 50-n that have transmitted the magnetic field mc by the multiplexed signal in one frame period before the current frame period are the certain threshold (5 mm) or larger.

When the determination result shows that the differences between the positions of the coils each are the certain threshold (5 mm) or larger, the signal control unit 56 causes the magnetic field mc by the multiplexed signal formed of a combined wave obtained by combining the first frequency f1 (=400 Hz) and the second frequency f2 (=800 Hz) to be transmitted at the next timing of transmitting magnetic fields from the transmitting coil 50-1 as No. 2, No. 3, . . . , and No. m.

In the second determination method, although the certain threshold of the difference between the positions of the transmitting coils is set to 5 mm, the threshold may be changed by user operation or an application program.

A threshold can be used to detect the direction of magnetic fields for each of the directions of the axes of the receiving coils 53-1 to 53-m that are the x, y, and z directions for detecting the magnetic fields transmitted from the transmitting coils 50-1 to 50-n.

The threshold may be set to a numerical value, for example, 5° that is the difference between the directions of the transmitting coils that have transmitted the magnetic field mc by the multiplexed signal in one frame period before the current frame period and the directions of the coils in the current frame period, and the threshold may be used to determine whether the magnetic field mc by the multiplexed signal is transmitted.

Generally, in a magnetic position detecting apparatus, positioning accuracy tends to be degraded when the distance between the transmitting coils 50-1 to 50-n and the receiving coils 53-1 to 53-m is large. For this reason, the threshold may be changed in accordance with the distance between the receiving coils 53-1 to 53-m as the antenna AT and the transmitting coils 50-1 to 50-n.

The following describes the operation of the apparatus 1 structured as described above.

In the first frame period (the first frame), the signal control unit 56 sequentially supplies the transmitting coils 50-1 to 50-n as No. n (n=1, 2, . . . , 30) with electric power having frequencies including the first frequency f1 (=400 Hz) and the second frequency f2 (=800 Hz) from the transmitter power supply 52 through the relay unit 51 at certain regular intervals by on/off-driving the relay unit 51.

With the supplied electric power, the transmitting coils 50-1 to 50-n sequentially transmit the magnetic field mc by the multiplexed signal formed of a combined wave obtained by combining the first frequency f1 (=400 Hz) and the second frequency f2 (=800 Hz), for example, at certain regular intervals as illustrated in FIG. 4. For example, each magnetic field mc by the multiplexed signal is transmitted in 10 ms with a varied amplitude in four wavelengths.

In order to estimate the influence of the magnitude of the magnetic fields due to eddy currents generated in external magnetic members (metals) existing in an environment in which magnetic fields are transmitted and received, the signal control unit 56 causes each of the transmitting coils 50-1 to 50-n to transmit the magnetic field mc by the multiplexed signal in the first frame period.

The receiving coils 53-1 to 53-m detect, in the x, y, and z directions, the magnetic fields mc sequentially transmitted from the transmitting coils 50-1 to 50-n, and generate the voltages corresponding to the magnitude of the magnetic fields in the x, y, and z directions, on both ends of each of the receiving coils 53-1 to 53-m.

The voltage detectors 54 each detect the voltage generated between both ends of each of the receiving coils 53-1 to 53-m and output the detection signal. The voltage detector 54 A/D-converts the detection signal in a chronological order, performs the FFT analysis on a sequence of the A/D-converted digital detection signals, and calculates the amplitude and the phase for each of the frequencies including the first frequency f1 (=400 Hz) and the second frequency f2 (=800 Hz).

In the first frame period, the transmitting coils 50-1 to 50-n each transmit the magnetic field mc having frequencies including the first frequency f1 (=400 Hz) and the second frequency f2 (=800 Hz) once, and the transmission of the magnetic fields is completed.

Every time the signal control unit 56 causes the transmitting coils 50-1 to 50-n to sequentially transmit the magnetic field, each voltage detector 54 A/D-converts each detection signal output from the voltage detector 54 in a chronological order, performs the FFT analysis on a sequence of the A/D-converted digital detection signals, and calculates the amplitude and the phase for each of the frequencies including the first frequency f1 (=400 Hz) and the second frequency f2 (=800 Hz).

In the first one frame period in which the magnetic field mc is generated by the multiplexed signal formed of a combined wave obtained by combining the first frequency f1 (=400 Hz) and the second frequency f2 (=800 Hz), the removing unit 57 obtains the difference in the voltages of the detection signals corresponding to the first frequency f1 (=400 Hz) and the second frequency f2 (=800 Hz) with respect to the receiving coils in the x direction, the receiving coils in the y direction, and the receiving coil's in the z direction.

The removing unit 57 calculates the magnitude of the magnetic fields due to eddy currents in the x, y, and z directions on the basis of the magnitude difference, and removes the calculated magnitude of the magnetic fields due to eddy currents in the x, y, and z directions from the magnitude of the magnetic fields transmitted from the transmitting coils 50-1 to 50-n, that is, the detection signals output from the voltage detectors 54.

Next, the signal control unit 56 causes the transmitting coils 50-1 to 50-n to sequentially transmit only the magnetic field h by the single signal having the second frequency f2 (=800 Hz) at certain regular intervals in each frame period after the first frame period, by driving the relay unit 51. For example, FIG. 5 illustrates transmission timing in the second frame period.

The receiving coils 53-1 to 53-m detect, in the x, y, and z directions, the magnetic fields h sequentially transmitted from the transmitting coils 50-1 to 50-n, and generate the voltages corresponding to the magnitude of the magnetic fields h on both ends of each of the receiving coils 53-1 to 53-m. The voltage detectors 54 each output the voltage detection signal corresponding to the voltage generated between both ends of each of the receiving coils 53-1 to 53-m.

The signal control unit 56 sets the certain threshold to a voltage, for example, corresponding to 2% of the amplitude of the second frequency f2 (=800 Hz) output from the voltage detectors 54 when the receiving coils 53-1 to 53-m have received the magnetic fields transmitted from the transmitting coils 50-1 to 50-n in one frame period before the current frame period. At this time, n×m pieces of thresholds are set.

In addition, the signal control unit 56 determines whether the magnitude of the magnetic fields detected by the receiving coils 53-1 to 53-m, that is, the amplitude output from each voltage detector 54 has changed to the certain threshold or higher. When the determination result shows that the amplitude output from the voltage detector 54 has changed to the certain threshold or higher, the signal control unit 56 causes the transmitting coils 50-1 to 50-n to transmit the magnetic field mc by the multiplexed signal in the next frame period.

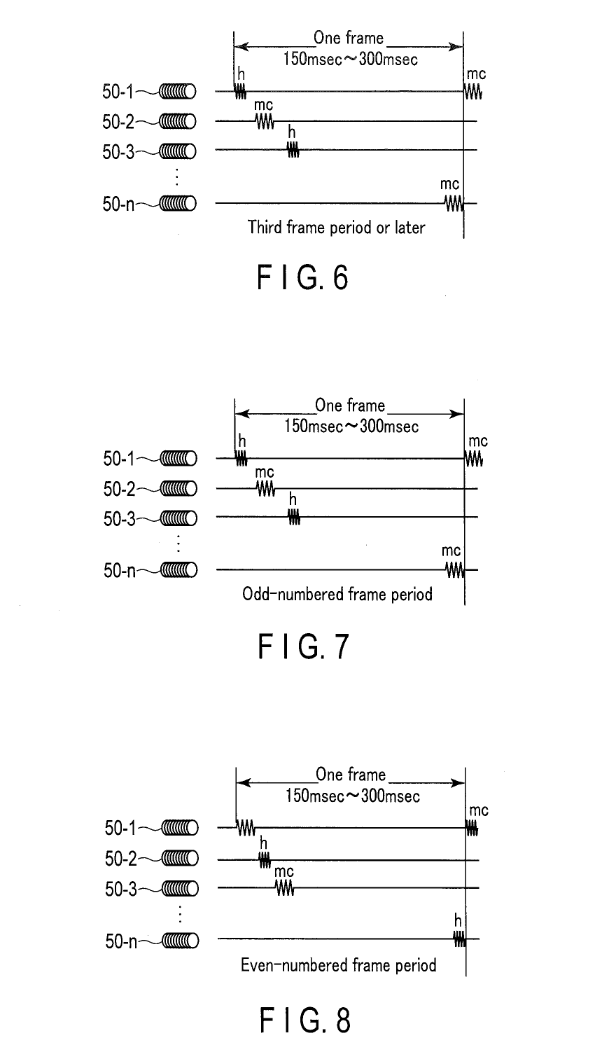

FIG. 6 illustrates exemplary transmission timing in the third frame period and the following frame periods. The example in FIG. 6 shows that, in one frame period before the current frame period, because the magnitude of the magnetic fields transmitted from the transmitting coils 50-2 and 50-n, among the magnetic fields detected by the receiving coils 53-1 to 53-m, has changed to the certain threshold or higher, the magnetic field mc by the multiplexed signal formed of the combined wave obtained by combining the first frequency f1 (=400 Hz) and the second frequency f2 (=800 Hz) is transmitted.

Specifically, the signal control unit 56 performs the first determination method, in other words, obtains the difference between the amplitude (first amplitude) of the second frequency f2 (=800 Hz) obtained when the receiving coils 53-1 to 53-m receive the magnetic fields and the amplitude (second amplitude) of the second frequency f2 (=800 Hz) obtained at the last time when the magnetic field mc by the multiplexed signal was transmitted and received, and determines whether the difference is the certain threshold or higher. The first amplitude is calculated by FFT-analyzing the detection signal output from the voltage detector 54 when the receiving coils 53-1 to 53-m receive the magnetic fields. The second amplitude is calculated by FFT-analyzing the detection signal output from the voltage detector 54 at the last time when the magnetic field mc by the multiplexed signal was transmitted and received, the detection signal corresponding to the magnitude of the magnetic fields.

When the determination result shows that the difference between the amplitudes is the certain threshold or higher, the signal control unit 56 causes the magnetic field mc by the multiplexed signal formed of the combined wave obtained by combining the first frequency f1 (=400 Hz) and the second frequency f2 (=800 Hz) to be transmitted at the transmission timing of the transmitting coils 50-1 to 50-n the amplitude difference of which is the certain threshold or higher.

The removing unit 57 calculates the amplitudes from which the influence of eddy currents in external magnetic members (metals) has been removed on the basis of the amplitudes and the phases of the first frequency f1 (=400 Hz) and the second frequency f2 (=800 Hz) when the magnetic field mc is generated by the multiplexed signal formed of the combined wave obtained by combining the first frequency f1 (=400 Hz) and the second frequency f2 (=800 Hz).

If the magnetic field h is generated, the removing unit 57 calculates the amplitudes from which the influence of eddy currents in external magnetic members (metals) has been removed on the basis of the width and the phase of the second frequency f2 (=800 Hz) and the influence ratio of the last eddy currents. The removing unit 57 removes the magnitude of the magnetic fields due to eddy currents in the x, y, and z directions.

The position detecting unit 55 detects the positions of the transmitting coils 50-1 to 50-n on the basis of the calculation results by the removing unit 57, thereby detecting the shape of the insertion member 20 of the endoscope apparatus 10 on the basis of the position of each of the transmitting coils 50-1 to 50-n; in one example, detecting the shape of the insertion member 20 inserted in a curve form or the like in a body cavity of a patient.

The signal control unit 56 may determine whether the voltage of the detection signal corresponding to the magnitude of the magnetic fields is the certain threshold or higher by the second determination method.

In the first embodiment, in the first frame period, the transmitting coils 50-1 to 50-n sequentially transmit the magnetic field mc by the multiplexed signal formed of the combined wave obtained by combining the first frequency f1 (=400 Hz) and the second frequency f2 (=800 Hz) at certain regular intervals, and in each frame period after the first frame period, when the voltage corresponding to the magnitude of the magnetic field detected by each of the receiving coils 53-1 to 53-m has changed to the certain threshold or higher, the transmitting coils 50-1 to 50-n each transmit the magnetic field mc by the multiplexed signal. When the magnetic field mc by the multiplexed signal is transmitted, the removing unit 57 removes the magnetic components generated on both ends of each of the receiving coils 53-1 to 53-m, the magnetic components being generated by external magnetic members (metals).

With this configuration, the magnetic field mc by the multiplexed signal is transmitted in the first frame period and a frame period in which the voltage corresponding to the magnitude of the magnetic field has changed to the certain threshold or higher. In the other frame periods, the transmitting coils 50-1 to 50-n transmit only the magnetic field h by the single signal having the second frequency f2 (=800 Hz). With this configuration, the time for transmitting the magnetic field can be shortened. The time for detecting the shape and other attributes of a medical device can be shortened without being affected by disturbance in magnetic fields due to eddy currents when the magnetic fields are transmitted and received.

For example, in a case where thirty transmitting coils 50-1 to 50-n disposed on the insertion member 20 of the endoscope 12 sequentially transmit the magnetic field mc by the multiplexed signal formed of the combined wave obtained by combining the first frequency f1 (=400 Hz) and the second frequency f2 (=800 Hz) in a transmission period of 10 ms with a varied amplitude in four wavelengths, the time for completing the transmission by all the transmitting coils 50-1 to 50-n is simply estimated to be 300 ms for one frame period. As a result, the frame rate is 3.33 frame/sm that is quite slow.

In the first embodiment, in the first frame period, the transmitting coils 50-1 to 50-n sequentially transmit the magnetic field mc by the multiplexed signal formed of the combined wave obtained by combining the first frequency f1 (=400 Hz) and the second frequency f2 (=800 Hz) and the magnitude of the magnetic fields affected by eddy currents is calculated. In each frame period after the first frame period, when the voltage of each received signal corresponding to the magnitude of the magnetic field at the position of each of the transmitting coils 50-1 to 50-n detected by each of the receiving coils 53-1 to 53-m has changed to the certain threshold or higher, each of the transmitting coils 50-1 to 50-n transmits the magnetic field by the multiplexed signal mc.

In the first embodiment, if the voltage of each received signal corresponding to the magnitude of the magnetic field at the position of each of the transmitting coils 50-1 to 50-n is not the certain threshold or higher, each of the transmitting coils 50-1 to 50-n transmits only the magnetic field h by the single signal having the second frequency f2 (=800 Hz).

With this configuration, the frame rate can be increased and the time for detecting the shape and other attributes of the medical device can be shortened without being affected by disturbance in magnetic fields due to eddy currents.

As a result, the shape of the insertion member 20, which is frequently operated to be curved, curved or bent in a body cavity (lumen) of a patient, can be detected accurately in a short time when the insertion member 20 of the endoscope apparatus 10 is inserted in the body cavity (lumen) of the patient as the subject to observe or treat an affected part or lesioned part in the body cavity (lumen).

### Second Embodiment

The following describes the second embodiment of the present invention with reference to the accompanying drawings. Note that the position detecting apparatus 1 for a medical device according to the second embodiment has the similar structure as that of the apparatus illustrated in FIG. 1, and therefore the different parts are described with reference to FIG. 1.

In the apparatus 1, similar to the first embodiment, a plurality of transmitting coils 50-1 to 50-n, for example, thirty transmitting coils (n=30) are disposed in the insertion member 20 of the endoscope apparatus 10 at certain intervals.

The receiving coils 53-1 to 53-m, for example, twelve receiving coils (m=12) that are components of the antenna AT are disposed outside a subject such as a patient.

The first frame period in consecutive frame periods is defined as an odd-numbered frame period. The second frame period is defined as an even-numbered frame period.

The receiving coils 53-1 to 53-m are disposed on the insertion member 20 of the endoscope apparatus 10 at certain regular intervals in an aligned manner. The transmitting coils 50-2, 50-4, . . . , and 50-30 (n=30) disposed at even-numbered positions from the distal end of the insertion member 20 are assumed to be disposed at first positions.

The transmitting coils 50-1, 50-3, . . . , and 50-29 (n=30) disposed at odd-numbered positions from the distal end of the insertion member 20 are assumed to be disposed at second positions.

On the basis of the positional relation of the transmitting coils 50-1 to 50-n, the signal control unit 56 causes the transmitting coils 50-1 to 50-n to transmit the magnetic field mc by a multiplexed signal formed of a combined wave obtained by combining at least two or more frequencies, for example, the first frequency f1 (=400 Hz) and the second frequency f2 (=800 Hz), by driving the relay unit 51.

In an odd-numbered frame period defined as one of the first frame periods in the consecutive frame periods, as illustrated in FIG. 7, the signal control unit 56 causes the transmitting coils 50-2, 50-4, . . . and 50-30 (n=30) that are disposed at even-numbered positions defined as the first positions among the arrangement positions of the transmitting coils 50-1 to 50-n to transmit the magnetic field mc by the multiplexed signal, and causes the transmitting coils 50-1, 50-3, . . . , and 50-29 (n=30) that are disposed at odd-numbered positions defined as the second positions to transmit only the magnetic field h by the second frequency f2 (=800 Hz).

In an even-numbered frame period defined as one of the second frame periods, as illustrated in FIG. 8, the signal control unit 56 causes the transmitting coils 50-1, 50-3, . . . and 50-29 (n=30) that are disposed at odd-numbered positions defined as the second positions to transmit the magnetic field mc by the multiplexed signal, and causes the transmitting coils 50-2, 50-4, . . . , and 50-30 (n=30) that are disposed at even-numbered positions defined as the first positions to transmit only the magnetic field h by the second frequency f2 (=800 Hz).

The following describes the operation of the apparatus 1 structured as described above.

In the first frame period, similar to the first embodiment, the signal control unit 56 causes the transmitting coils 50-1 to 50-n to sequentially transmit the magnetic field mc by the multiplexed signal formed of the combined wave obtained by combining the first frequency f1 (=400 Hz) and the second frequency f2 (=800 Hz) at certain regular intervals.

Every time the transmitting coils 50-1 to 50-n sequentially transmit the magnetic field, the voltage detectors 54 A/D-convert the voltage detection signals in a chronological order, perform the FFT analysis on a sequence of the A/D-converted digital detection signals, and calculate the amplitude and the phase for each of the frequencies including the first frequency f1 (=400 Hz) and the second frequency f2 (=800 Hz).

In the first one frame period in which the magnetic field mc is generated by the multiplexed signal formed of a combined wave obtained by combining the first frequency f1 (=400 Hz) and the second frequency f2 (=800 Hz), the removing unit 57 calculates the amplitudes from which the influence of eddy currents in external magnetic members (metals) has been removed on the basis of the amplitudes and the phases of, for example, the first frequency f1 (=400 Hz) and the second frequency f2 (=800 Hz) for each of the receiving coils 53-1, . . . , and 53-n. The amplitude determination methods are publicly known. For example, refer to Jpn. PCT National Publication No. 2003-513260 and Jpn. PCT National Publication No. 2006-523473. The amplitude calculated for each frequency, along with a phase, is sent to the position detecting unit 55 included in the inserted shape estimating unit.

The operation of the position detecting unit 55 is the same as that in the first embodiment.

In an even-numbered frame period defined as one of the second frame periods, as illustrated in FIG. 8, the signal control unit 56 causes the transmitting coils 50-1, 50-3, . . . and 50-29 (n=30) that are disposed at odd-numbered positions defined as the second positions to transmit the magnetic field mc by the multiplexed signal, and causes the transmitting coils 50-2, 50-4, . . . , and 50-30 (n=30) that are disposed at even-numbered positions defined as the first positions to transmit only the magnetic field h by the second frequency f2 (=800 Hz). In other words, the transmitting coils 50-1, 50-3, . . . , and 50-29 (n=30) disposed at the odd-numbered positions sequentially transmit the magnetic field mc by the multiplexed signal formed of the combined wave obtained by combining the first frequency f1 (=400 Hz) and the second frequency f2 (=800 Hz) at certain regular intervals.

The transmitting coils 50-2, 50-4, . . . , and 50-30 (n=30) disposed at the even-numbered positions sequentially transmit only the magnetic field h by the second frequency f2 (=800 Hz) at certain regular intervals.

The transmission of the magnetic fields mc and h from the transmitting coils 50-1, 50-3, . . . , and 50-29 (n=30) disposed at the odd-numbered positions and the transmitting coils 50-2, 50-4, . . . , and 50-30 (n=30) disposed at the even-numbered positions may be performed in the order of the transmitting coils 50-1 to 50-n.

The receiving coils 53-1 to 53-m detect, in the x, y, and z directions, the magnetic fields mc and h sequentially transmitted from the transmitting coils 50-1, 50-3, . . . , and 50-29 (n=30) disposed at the odd-numbered positions and the transmitting coils 50-2, 50-4, . . . , and 50-30 (n=30) disposed at the even-numbered positions, respectively, and generate the voltages corresponding to the magnitude of the magnetic fields in the x, y, and z directions on both ends of each of the receiving coils 53-1 to 53-m.

The voltage detectors 54 each detect the voltage generated between both ends of each of the receiving coils 53-1 to 53-m, A/D-convert the detection signal in a chronological order, perform the FFT analysis on a sequence of the A/D-converted digital detection signals, calculate the amplitude and the phase for each of the frequencies including the first frequency f1 (=400 Hz) and the second frequency f2 (=800 Hz), and transmit the resultant amplitude and phase to the removing unit 57.

The removing unit 57 calculates the amplitudes from which the influence of eddy currents in external magnetic members (metals) has been removed on the basis of the amplitudes and the phases of, for example, the first frequency f1 (=400 Hz) and the second frequency f2 (=800 Hz) for each of the receiving coils 53-1, . . . , and 53-m. The amplitude determination methods are publicly known. For example, refer to Jpn. PCT National Publication No. 2003-513260 and Jpn. PCT National Publication No. 2006-523473. The amplitude calculated for each frequency, along with a phase, is sent to the position detecting unit 55 included in the inserted shape estimating unit.

In the calculation in the removing unit 57 in an even-numbered frame period, with respect to the transmitting coils 50-1, 50-3, . . . , and 50-29 (n=30) disposed at the odd-numbered positions as the second positions, the amplitudes and the phases of the first frequency f1 (=400 Hz) and the second frequency f2 (=800 Hz) of the received signal in the current frame period are used.

With respect to the transmitting coils 50-2, 50-4, . . . , and 50-30 (n=30) disposed at the even-numbered positions as the first positions, the amplitudes and the phases of the first frequency f1 (=400 Hz) of the received signal in one frame period before the current frame period and the second frequency f2 (=800 Hz) of the received signal in the current frame period are used.

The operation of the position detecting unit 55 is the same as that in the first embodiment.

In an odd-numbered frame period, the signal control unit 56 sequentially supplies the transmitting coils 50-2, 50-4, . . . , and 50-30 (n=30) disposed at the even-numbered positions as the first positions with electric power having frequencies including the first frequency f1 (=400 Hz) and the second frequency f2 (=800 Hz) from the transmitter power supply 52 through the relay unit 51 at certain regular intervals by on/off-driving the relay unit 51.

With the supplied electric power, the transmitting coils 50-2, 50-4, . . . , and 50-30 (n=30) disposed at the even-numbered positions sequentially transmit the magnetic field mc by the multiplexed signal formed of the combined wave obtained by combining the first frequency f1 (=400 Hz) and the second frequency f2 (=800 Hz), for example, at certain regular intervals as illustrated in FIG. 7. For example, each magnetic field mc by the multiplexed signal is transmitted in 10 ms with a varied amplitude in four wavelengths.

The receiving coils 53-1 to 53-m detect, in the x, y, and z directions, the magnetic fields mc and h sequentially transmitted from the transmitting coils 50-1 to 50-n, and generate the voltages corresponding to the magnitude of the magnetic fields in the x, y, and z directions on both ends of each of the receiving coils 53-1 to 53-m.

The voltage detectors 54 each detect the voltage generated between both ends of each of the receiving coils 53-1 to 53-m, A/D-convert the detection signal in a chronological order, perform the FFT analysis on a sequence of the A/D-converted digital detection signals, calculate the amplitude and the phase for each of the frequencies including the first frequency f1 (=400 Hz) and the second frequency f2 (=800 Hz), and transmit the resultant amplitude and phase to the removing unit 57.

The removing unit 57 calculates the amplitudes from which the influence of eddy currents in external magnetic members (metals) has been removed on the basis of the amplitudes and the phases of, for example, the first frequency f1 (=400 Hz) and the second frequency f2 (=800 Hz) for each of the receiving coils 53-1, . . . , and 53-m. The amplitude determination methods are publicly known. For example, refer to Jpn. PCT National Publication No. 2003-513260 and Jpn. PCT National Publication No. 2006-523473. The amplitude calculated for each frequency, along with a phase, is sent to the position detecting unit 55 included in the inserted shape estimating unit.

In the calculation in the removing unit 57 in an odd-numbered frame period, with respect to the transmitting coils 50-2, 50-4, . . . , and 50-30 (n=30) disposed at the odd-numbered positions as the first positions, the amplitudes and the phases of the first frequency f1 (=400 Hz) and the second frequency f2 (=800 Hz) of the received signal in the current frame period are used.

With respect to the transmitting coils 50-1, 50-3, . . . , and 50-29 (n=30) disposed at the even-numbered positions as the second positions, the amplitudes and the phases of the first frequency f1 (=400 Hz) of the received signal in one frame period before the current frame period and the second frequency f2 (=800 Hz) of the received signal in the current frame period are used.

The operation of the position detecting unit 55 is the same as that in the first embodiment.

In the second embodiment, in an odd-numbered frame period, the transmitting coils 50-2, 50-4, . . . , and 50-30 (n=30) disposed at the even-numbered positions each transmit the magnetic field mc by the multiplexed signal, and the transmitting coils 50-1, 50-3, . . . , and 50-29 (n=30) disposed at the odd-numbered positions each transmit only the magnetic field h by the second frequency f2 (=800 Hz).

In an even-numbered frame period, the transmitting coils 50-1, 50-3, . . . , and 50-29 (n=30) disposed at the odd-numbered positions each transmit the magnetic field mc by the multiplexed signal, and the transmitting coils 50-2, 50-4, . . . , and 50-30 (n=30) disposed at the even-numbered positions each transmit only the magnetic field h by the second frequency f2 (=800 Hz).

With this configuration, transmission of the magnetic field mc by the multiplexed signal can be completed in a short time without transmitting the magnetic field mc by the multiplexed signal from all the transmitting coils 50-1 to 50-n. Consequently, the frame rate can be increased and the time for detecting the shape and other attributes of the medical device can be shortened without being affected by disturbance in magnetic fields due to eddy currents.

### Third Embodiment

The following describes the third embodiment of the present invention with reference to the accompanying drawings. Note that the position detecting apparatus 1 for a medical device according to the third embodiment has the similar structure as that of the apparatus illustrated in FIG. 1, and therefore the different parts are described with reference to FIG. 1.

In the apparatus 1, similar to the first embodiment, a plurality of transmitting coils 50-1 to 50-n, for example, thirty transmitting coils (n=30) are disposed on the insertion member 20 of the endoscope apparatus 10 at certain intervals.

The receiving coils 53-1 to 53-m, for example, twelve receiving coils (m=12) that are components of the antenna AT are disposed outside a subject such as a patient.

The signal control unit 56 causes, among the transmitting coils 50-1 to 50-n, first coils disposed on the distal end side of the insertion member 20, for example, the five transmitting coils 50-1 to 50-5 from the distal end, to transmit the magnetic field mc by the multiplexed signal through all the frame periods.

The signal control unit 56 causes second coils other than the first coils, that is, the transmitting coils 50-6 to 50-n disposed at the sixth and the following positions from the distal end of the insertion member 20, to transmit the magnetic field mc by the multiplexed signal once per several frame periods.

In the third embodiment, the first coils disposed on the distal end side of the insertion member 20 of the endoscope apparatus 10, for example, the five transmitting coils 50-1 to 50-5 from the distal end, transmit the magnetic field mc by the multiplexed signal in each frame period, and therefore transmission of the magnetic field mc by the multiplexed signal can be completed in a short time without transmitting the magnetic field mc by the multiplexed signal from all the transmitting coils 50-1 to 50-n. With this configuration, the frame rate can be increased and the time for detecting the shape and other attributes of the medical device can be shortened without being affected by disturbance in magnetic fields due to eddy currents.

The first coils disposed on the distal end side of the insertion member 20 of the endoscope apparatus 10, for example, the five transmitting coils 50-1 to 50-5 from the distal end, transmit the magnetic field mc by the multiplexed signal in each frame period, and therefore the shape and other attributes of the insertion member 20 on the distal end can be detected in a short time. The insertion member 20 of the endoscope apparatus 10 is frequently operated to be curved or bent when being inserted in a body cavity (lumen) of a patient as the subject to observe or treat an affected part or lesioned part in the body cavity (lumen), and therefore it is important that the shape of the insertion member 20 curved or bent in the body cavity (lumen) of the patient can be detected accurately in a short time. The apparatus 1 can accomplish such technical objective.

It is to be noted that the present invention is not restricted to the foregoing embodiments, and it can be embodied by modifying constituent elements without departing from the scope thereof in an embodying stage. Furthermore, various kinds of inventions can be formed by appropriately combining a plurality of constituent elements disclosed in the foregoing embodiments. For example, some constituent elements can be eliminated from all the constituent elements described in the foregoing embodiments. Moreover, constituent elements in the different embodiments can be appropriately combined.

What is claimed is:

1. A position detecting apparatus for a medical device, comprising:
a transmission unit configured to transmit a magnetic field in each of a plurality of frame periods;
a reception unit configured to detect the magnetic field transmitted from the transmission unit and output a detection signal corresponding to a position of the transmission unit; and
a signal control unit configured to
cause the transmission unit to transmit the magnetic field by a multiplexed signal including at least two or more different frequencies in a certain frame period among the frame periods, and
cause the transmission unit to transmit the magnetic field by the multiplexed signal in the frame periods other than the certain frame period when the detection signal output from the reception unit has changed to a certain threshold or higher.

2. The position detecting apparatus for a medical device according to claim 1, further comprising a removing unit configured to remove magnetic components generated by an external magnetic member from the detection signal output from the reception unit when the transmission unit transmits the magnetic field by the multiplexed signal.

3. The position detecting apparatus for a medical device according to claim 1, wherein
the signal control unit
causes the transmission unit to transmit the magnetic field by the multiplexed signal in a first frame period in the frame periods, and
causes the transmission unit to transmit the magnetic field by the multiplexed signal in each frame period after the first frame period when the detection signal output from the reception unit has changed to the certain threshold or higher.

4. The position detecting apparatus for a medical device according to claim 1, wherein
the at least two or more frequencies include a first frequency and a second frequency higher than the first frequency, and
the signal control unit causes the transmission unit to transmit only the magnetic field by the second frequency when the detection signal output from the reception unit is not the certain threshold or higher.

5. The position detecting apparatus for a medical device according to claim 1, wherein
the transmission unit includes a plurality of coils, and
the reception unit detects the magnetic field transmitted from each of the coils and outputs the detection signal corresponding to each of positions of the coils.

6. The position detecting apparatus for a medical device according to claim 1, wherein
the reception unit outputs the detection signal having magnitude corresponding to a position of the transmission unit.

7. The position detecting apparatus for a medical device according to claim 5, wherein
the coils are disposed in an aligned manner,
the signal control unit
causes, among the coils, a first coil disposed on a distal end to transmit the magnetic field by the multiplexed signal in each of the frame periods, and
causes a second coil other than the first coil to transmit the magnetic field by the multiplexed signal in each term of several frame periods.

8. An endoscope apparatus comprising:
the position detecting apparatus for a medical device according to claim 1; and
a flexible insertion member configured to be inserted in a subject, wherein
the transmission unit is disposed on the insertion member.

9. A position detecting apparatus for a medical device, comprising:
a transmission unit including a plurality of coils and configured to transmit a magnetic field in each of a plurality of frame periods;
a reception unit configured to detect the magnetic field transmitted from the transmission unit and output a detection signal corresponding to each of positions of the coils; and
a signal control unit configured to cause the transmission unit to transmit the magnetic field by a multiplexed signal including at least two or more different frequencies from each of the coils on the basis of a positional relation of the coils.

10. The position detecting apparatus for a medical device according to claim 9, further comprising a removing unit configured to remove magnetic components generated by an external magnetic member from the detection signal output from the reception unit when the magnetic field by the multiplexed signal is transmitted.

11. The position detecting apparatus for a medical device according to claim 9, wherein
the at least two or more frequencies include a first frequency and a second frequency higher than the first frequency,
the signal control unit
causes each of the coils that are disposed at first positions among arrangement positions of the coils to transmit the magnetic field by the multiplexed signal in a first frame period in the frame periods, and causes each of the coils that are disposed at second positions to transmit only the magnetic field by the second frequency, and
causes each of the coils that are disposed at the second positions among the arrangement positions of the coils to transmit the magnetic field by the multiplexed signal in a second frame period that is different from the first frame period, and causes each of the coils disposed at the first positions to transmit only the magnetic field by the second frequency.

12. The position detecting apparatus for a medical device according to claim 11, wherein
the first frame period in the frame periods is an odd-numbered frame period,
the second frame period in the frame periods is an even-numbered frame period,
the plurality of coils are disposed in an aligned manner,
the coils disposed at first positions are disposed at even-numbered positions, and
the coils disposed at second positions are disposed at odd-numbered positions.

13. The position detecting apparatus for a medical device according to claim 9, wherein
the coils are disposed in an aligned manner,
the signal control unit
causes, among the coils, a first coil disposed on a distal end side to transmit the magnetic field by the multiplexed signal in each of the frame periods, and
causes a second coil other than the first coil to transmit the magnetic field by the multiplexed signal in each term of several frame periods.

14. The position detecting apparatus for a medical device according to claim 9, wherein
the reception unit outputs the detection signal having magnitude corresponding to a position of the transmission unit.

15. An endoscope apparatus comprising:
the position detecting apparatus for a medical device according to claim 9; and
a flexible insertion member configured to be inserted in a subject, wherein
the transmission unit is disposed on the insertion member.

16. A position detecting method for a medical device, comprising:
transmitting a magnetic field from a transmission unit in each of a plurality of frame periods;
detecting the magnetic field by a reception unit and outputting a detection signal corresponding to a position of the transmission unit;
causing the transmission unit to transmit the magnetic field by a multiplexed signal including at least two or more different frequencies in a certain frame period among the frame periods; and
causing the transmission unit to transmit the magnetic field by the multiplexed signal in the frame periods other than the certain frame period when the detection signal output from the reception unit has changed to a certain threshold or higher.

17. A position detecting method for a medical device, comprising:
transmitting a magnetic field from a plurality of coils in each of a plurality of consecutive frame periods;
receiving the transmitted magnetic field by a reception unit and outputting a detection signal corresponding to each of positions of the coils; and
causing, by a signal control unit, the transmission unit to transmit the magnetic field by a multiplexed signal including at least two or more different frequencies from each of the coils on the basis of a positional relation of the coils.
